(19) United States
(12) Patent Application Publication
(10) Pub. No.: US 2021/0001018 A1
(43) Pub. Date: Jan. 7, 2021

(54) METHOD AND DEVICE FOR SURGERY

(71) Applicant: HERIOT EYECARE PTY. LTD., Melbourne, Victoria (AU)

(72) Inventors: Wilson J. Heriot, Melbourne, Victoria (AU); Shadi Houshyar, Melbourne, Victoria (AU); Bang Viet Bui, Melbourne, Victoria (AU); Andrew Bernard Metha, Melbourne, Victoria (AU)

(21) Appl. No.: 16/979,386

(22) PCT Filed: Mar. 8, 2019
(86) PCT No.: PCT/AU2019/000035

§ 371 Date: Sep. 9, 2020

(30) Foreign Application Priority Data
Mar. 9, 2018 (AU) 2018900776

(51) Int. Cl. A61M 1/00; A61F 9/007

(52) U.S. Cl. CPC A61M 1/008; A61F 9/00736; A61M 2210/0612; A61M 2202/04; A61M 1/0023

(57) ABSTRACT

The invention provides devices, methods and kits for surgery. More specifically, the invention provides a device for surgery comprising: a body comprising a handle; and one or hydrophilic extension or probe, wherein the device is adapted for removal of hydrophilic fluid. A method is also provided for removal of hydrophilic fluid, the method comprising: contacting the hydrophilic fluid with one or more hydrophilic extension or probe to thereby remove the hydrophilic fluid.

### FIELD OF THE INVENTION

The present invention relates to a method and device for surgery. More particularly, this invention relates to a method and device for surgery using one or more extension or probe to assist in the removal of fluid.

### BACKGROUND TO THE INVENTION

As described in the inventor's earlier patent publication WO2014/110624, the publication of PCT/AU2014/000023, in the treatment of retinal detachment, it can be useful to remove water, fluid or hydrophilic fluid.

In retinal detachment and other eye surgeries there remains a need to withdraw fluid such as, retinal fluid, from the junction of the retinal tear. In retinal detachment the removal of the fluid is conducted prior to tear dehydration to accomplish effective thermofusion.

There remains a need for alternative techniques for the removal of fluid from the eye.

The reference to any prior art in this specification is not, and should not be taken as, an acknowledgement or any form of suggestion that the prior art forms part of the common general knowledge.

### OBJECT OF THE INVENTION

It is a preferred object of the embodiments of the present invention to provide an apparatus that addresses or at least ameliorates one or more of the aforementioned problems of the prior art and/or provides a useful commercial alternative.

### SUMMARY OF THE INVENTION

Generally, embodiments of the present invention relate to methods and devices for surgery. The methods and device may find particular application to the eye.

In a broad form, the invention relates to use of one or more extension or probe to assist in the removal of fluid.

In a first form, although it need not be the only or indeed the broadest form, the invention resides in a method for removal of hydrophilic fluid, the method including:
contacting the hydrophilic fluid with one or more hydrophilic extension or probe to thereby remove the hydrophilic fluid.

In a first particular embodiment of the first form the removal is by one or more of cohesive attraction, adhesion and capillary action. The removal may be specific or substantially specific for hydrophilic fluid. The specificity for hydrophilicity may be over hydrophobicity or oleophilicity.

According to the first particular embodiment of the first form, no extraneous or additional force or energy is applied. The removal is by intrinsic forces.

In a second particular embodiment of the first form, the device applies aspiration to assist in removal of the hydrophilic fluid.

According to the second particular embodiment of the first form, extraneous or additional force or energy in the form of aspiration is applied.

In a second form, the invention resides in a device for surgery comprising:
a body comprising a handle; and
one or hydrophilic extension or probe.

The device for surgery of the second form may be for removal of hydrophilic fluid.

In a first particular embodiment of the second form the device removes the hydrophilic fluid by one or more of cohesive attraction, adhesion and capillary action. The removal may be specific or substantially specific for the hydrophilic fluid.

According to the first particular embodiment of the second form, the device does not apply any extraneous or additional force or energy.

In a second particular embodiment of the second form, the device applies aspiration to assist in removal of the hydrophilic fluid.

According to the second particular embodiment of the second form, extraneous or additional force or energy in the form of aspiration is applied.

In a third form, the invention resides in a method for manufacturing a device for surgery comprising:
disposing one or more one or hydrophilic extension or probe on a body comprising a handle.

In a fourth form, the invention resides in a kit for surgery comprising the device of the second form. The kit may further comprise an aspiration device and/or instructions for use. The aspiration device is any device that can apply an aspirating force. The aspiration device may be or may comprise the Alcon Constellation device, the Synergetics VersaVIT 2.0 Vitrectomy System, any other suitable commercially available device or one or more component thereof.

In one embodiment of any above form, the one or more of cohesive attraction, adhesion and capillary action is between the one or more hydrophilic extension or probe and the hydrophilic fluid.

In another embodiment of any above form, the one or more of cohesive attraction, adhesion and capillary action may be the result of non-covalent bonding. The non-covalent bonding may comprise one or more of electrostatic interactions, van der Waals forces, π-effects and the hydrophilic effects. The hydrophilic attraction may be charge-polarized and/or capable of hydrogen bonding.

In yet another embodiment of any above form, the removal by one or more of cohesive attraction, adhesion and capillary action may further comprise one or more of wicking and mopping.

In one embodiment of any above form the one or more hydrophilic extension or probe comprises one or more of polylactic acid (PLA); polyvinyl acetate (PVA) and chitosan. The chitosan may be from a crustacean. The one or more hydrophilic extension or probe may be obtained from the School of Fashion & Textiles or Centre for Materials Innovation and Future Fashion (CMIFF) at RMIT University, Melbourne, Australia.

In one embodiment of any above form, the one or more hydrophilic extension or probe may comprise a large surface area. The one or more hydrophilic extension or probe may comprise one or more hydrophilic fibres; mesh; or open weave. The fibres may be cross-linked. The open-weave may be coral-like. The extension or probe may be like the brushes of a paintbrush.

The one or more extension or probe may be produced by melt extrusion and/or wet spinning.

The one or more extension or probe may absorb hydrophilic fluid.

The one or more extension or probe may be of any suitable dimension, such as width, length and thickness.

In an embodiment, the one or more extension or probe may comprise a diameter from 50 micron to 700 microns.

In another embodiment, the one or more extension or probe may comprise a diameter from 1 micron to 50 microns, or from 5 micron to 40 microns, or from 10 micron to 25 microns.

In a particular embodiment, the one or more extension or probe may comprise a diameter of up to 50 microns. The one or more extension or probe may comprise a diameter of 1; 2; 3; 4; 5; 6; 7; 8; 9; 10; 11; 12; 13; 14; 15; 16; 17; 18; 19; 20; 21; 22; 23; 24; 25; 26; 27; 28; 29; 30; 35; 40; 41; 42; 43; 44; 45; 46; 47; 48; 49; or 50 microns.

In a preferred embodiment of any of the above forms, the one or more hydrophilic fibres comprise one or more nanofibers and/or one or more microfibres. It will be appreciated that the fibres may be provided as one or more bundles.

In another embodiment of any of the above forms, the handle may be for gripping by a clinician or by a robot. The clinician may be an ophthalmologist or a surgeon.

In still another embodiment of any one of the above forms, the one or more hydrophilic extension or probe may be disposed to make contact with hydrophilic fluid in a body part. The body part may comprise the eye or any suitable tissue plane and/or organ. The hydrophilic fluid may be in the form of a droplets or film. The droplets may be microdroplets. The film may be a fine film.

In yet another embodiment of any one of the above forms, the handle may be at a proximal end of the device.

In another embodiment of any one of the above forms, the one or more hydrophilic extension or probe may be at a distal end of the device.

In still another embodiment of any one of the above forms, the device may comprise a bore. The bore may allow passage of air for aspiration of fluid from the eye. The bore may comprise an internal bore. The fluid may comprise or consist of the hydrophilic fluid such as, subretinal fluid.

In yet another embodiment of any one of the above forms, the fluid removed comprises a meniscus remaining after active aspiration. The active aspiration may be with a standard soft-tip flute needle.

In still another embodiment of any one of the above forms, the method or device may be for eye surgery.

In another embodiment of any one of the above forms, the fluid is removed by the one or more hydrophilic extension or probe conforming to the junction between the retina and the retinal pigmented epithelium (RPE). The fluid may be removed in preparation for thermofusion and/or repair of retinal detachment.

In still another embodiment of any one of the above forms, the fluid removed may be from between edges of the conjunctiva. The fluid may be removed from the conjunctiva in preparation for thermofusion for glaucoma surgery.

In yet another embodiment of any one of the above forms, the fluid removed may be moisture from an intraocular lens (IOL) or other surface.

In another embodiment of any one of the above forms, the bore may comprise 5; 6; 7; 8; 9; 10; 11; 12; 13; 14; 15; 16; 17; 18; 19; 20; 21; 22; 23; 24; 25; 26; 27; 28; 29; 30; 35; 40 or greater than gauge diameter.

In yet another embodiment of any one of the above forms, the method and device may comprise a flute needle.

In still another embodiment of any one of the above forms, the method and device may comprise a leak free connection. The leak free connection may comprise a Luer fitting, a Luer lock or a Luer taper. The device may further comprise an internal channel connecting a male Luer terminal to an exit port on the side of the device or of the handle. The exit port may be disposed in a depression. The exit port may be closed by a finger or robotic component to prevent flow of fluid or gas from the eye. Removal of the finger or robotic component allows egress of fluid along the needle and through the exit port.

According to any one of the above forms, an infusion of gas or fluid may be provided to the eye to maintain the intraocular pressure. The intraocular pressure may be maintained above atmospheric.

In still another embodiment of any one of the above forms, the method and device may comprise a collection chamber. The collection chamber may be in fluid communication with the one or more hydrophilic extension or probe.

The collection chamber may comprise a hydrophilic material. The hydrophilic material may comprise a mesh. The mesh may be porous. The mesh may comprise one or more of polylactic acid or polylactide (PLA); polycaprolactone (PCL); poly(vinyl acetate) (PVA); or mixtures thereof.

In still another embodiment of any one of the above forms, the method and device may comprise a mesh. The mesh may be disposed in the bore.

Further forms, aspects and/or features of the present invention will become apparent from the following detailed description.

### BRIEF DESCRIPTION OF THE DRAWINGS

In order that the invention may be readily understood and put into practical effect, reference will now be made to embodiments of the present invention with reference to the accompanying drawings, wherein like reference numbers refer to identical elements. The drawings are provided by way of example only, wherein:

FIG. 1A (top left), 1B (top right), 1C (bottom left) and 1D (bottom right) illustrate how a standard flute can not contact or aspirate subretinal fluid or the meniscus but the present invention can access and remove the subretinal fluid and meniscus.

FIG. 2 is flowchart showing a method according to one embodiment of the invention.

Skilled addressees will appreciate that elements in the drawings are illustrated for simplicity and clarity and have not necessarily been drawn to scale. For example, the relative dimensions of some elements in the drawings may be distorted to help improve understanding of embodiments of the present invention.

### DETAILED DESCRIPTION OF THE INVENTION

The present invention relates to a method and device for surgery. Embodiments of the present invention relate to a method and device for surgery using one or more extension or probe to assist in the removal of hydrophilic fluid.

While the invention will be described with reference to the removal of subretinal fluid, it is not so limited. The device and method of the invention may find application throughout the body, tissue planes or organs.

As used herein the terms “hydrophilic” means a molecule or portion of a molecule whose interactions with water and other polar substances are more thermodynamically favourable than their interactions with oil or other hydrophobic solvents.

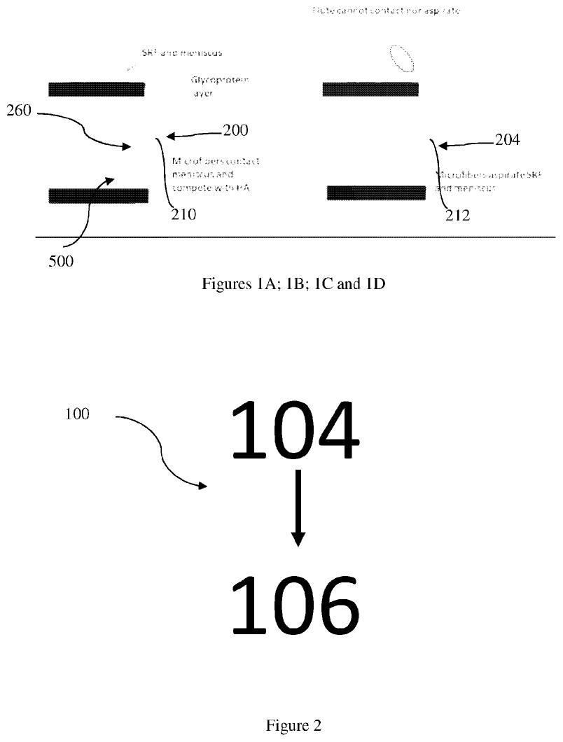

Through diligent research, the present inventor has sought an improved method and device for the removal of hydrophilic fluid such as, sub-retinal fluid from the eye, see FIG. 1A. The inventor has found that standard techniques and equipment is ineffective in removing the sub-retinal fluid and meniscus (see FIG. 1B). A standard flute needle can not contact or aspirate the sub-retinal fluid.

The invention is at least partly predicated on the unexpected discovery that one or more hydrophilic extension or probe can be used to remove sub-retinal fluid with minimal or no aspiration of the fluid.

A flowchart showing one embodiment of a method 100 according to the invention is shown in FIG. 2. In step 104 hydrophilic fluid such as, sub-retinal fluid in the eye, is contacted with one or more hydrophilic extension or probe disposed on an aspiration device.

The contact between the one or more hydrophilic extension or probe and the hydrophilic fluid results from or by one or more of absorption, cohesive attraction, adhesion and capillary action. The contact and resulting absorption, cohesive attraction, adhesion and/or capillary action simplifies the removal. When the one or more hydrophilic extension or probe is removed or withdrawn, the sub-retinal fluid is also removed or withdrawn.

Advantageously, the removal or withdrawal is specific or substantially specific for the hydrophilic fluid.

The one or more of absorption, cohesive attraction, adhesion and capillary action may be the result of non-covalent bonding such as electrostatic interactions, van der Waals forces, π-effects and the hydrophobic effect. The hydrophilic attraction may be charge-polarized and/or capable of hydrogen bonding.

As will be readily appreciated by a skilled person, this removal or withdrawal by one or more of absorption, cohesive attraction, adhesion and capillary action may further comprise one or more of wicking and mopping.

The contact and resulting absorption, cohesive attraction, adhesion and/or capillary action means that no extraneous or additional force or energy needs to be applied to achieve removal or withdrawal. The absorption, cohesive attraction, adhesion and/or capillary action are intrinsic forces.

As will be readily appreciated by a skilled person, the collection chamber, is in fluid communication with the one or more hydrophilic extension or probe such that when the one or more hydrophilic extension or probe is in contact with hydrophilic fluid, the fluid flows along the probe and is captured in the collection chamber. The capture may be by, or aided by, one or more hydrophilic material. The hydrophilic may comprise a mesh. The capture of the fluid in the collection chamber, as will be appreciated by the skilled person, is advantageous as it reduces clogging of the one or more hydrophilic extension or probe and associated adverse effects on the flow of the fluid.

Method 100 is also shown to include the optional step of aspirating 106. The aspiration may be of the hydrophilic fluid such as, subretinal fluid and/or other extraneous fluid such as BSS.

The skilled person will readily appreciate when aspiration is used, extraneous or additional force or energy is required in addition to the cohesive attraction, adhesion and/or capillary action.

The invention also provides a device for eye surgery. One embodiment of a device 200 according to the invention is shown in FIGS. 1C and 1D. Device 200 comprises a body 210, a collection chamber 213 (not shown), comprising a handle 214 (not shown) and one or hydrophilic extension or probe 260 in the form of fibres.

As will be readily appreciated by the skilled person, the one or more hydrophilic extension or probe 260 may be selected to have a large surface area. In addition to the one or more fibres shown in FIGS. 1C and 1D, the one or more extension or probe 260 may comprise one or more cross-linked fibre; mesh; or an open weave. The open-weave may be coral-like. The extension or probe 260 may be like the brushes of a paintbrush.

From the teaching herein, a skilled person is readily able to select a suitable material for the one or more hydrophilic extension or probe 260.

Provision of handle 214 is advantageous for gripping by a clinician or by a robot. The clinician may be an ophthalmologist or a surgeon. Handle 214 is disposed at a proximal end of the device. In some embodiments, the collection chamber 213 is disposed intermediate the handle 214 and the proximal end of the device.

As shown in FIGS. 1C and 1D, the one or more hydrophilic extension or probe 260 is/are disposed to make contact with hydrophilic fluid in an eye. As shown in FIGS. 1C and 1D, the one or more hydrophilic extension or probe 260 is/are disposed at a tip 212 a distal end 204 of device 200.

The one or more extension or probe 260 may be produced by melt extrusion and/or wet spinning. In the embodiment shown, the one or more extension or probe 260 comprises a plurality of fibres comprised of polylactic acid (PLA); polyvinyl acetate (PVA) and/or chitosan. The chitosan may be from a crustacean,

From the teaching herein a skilled person is readily able to select suitable dimensions for the one or more extension or probe 260. For example, when provided as microfibers, the one or more extension or probe 260 may comprise a diameter of 150 micron to 700 micron. The dimensions can be tailored by changing die and/or drawing method.

Device 200 comprises an internal bore 220 (not shown) which allows passage of air for aspiration of fluid from the eye. The fluid may comprise or consist of the hydrophilic fluid 500. As will be appreciated from the above explanation of removal with intrinsic force along, in other embodiments device 200 is not required to have a bore or to provide aspiration.

From the teaching herein, a skilled person is readily able to select a suitable bore. The bore may comprise 5; 6; 7; 8; 9; 10; 11; 12; 13; 14; 15; 16; 17; 18; 19; 20; 21; 22; 23; 24; 25; 26; 27; 28; 29; 30; 35; 40 or greater than gauge diameter.

Through provision of bore 220, device 200 may comprise a flute needle.

Device 200 is able to aspirate the sub-retinal fluid and/or meniscus as the one or more hydrophilic extension or probe 260 is spread or spreads over the sub-retinal fluid.

The fluid removed may comprise a meniscus remaining after active aspiration with a standard soft-tip flute needle.

The fluid may be removed by the one or more hydrophilic extension or probe conforming to the junction between the retina and the retinal pigmented epithelium (RPE). While not limited thereto, the fluid may be removed in preparation for thermofusion and/or repair of retinal detachment.

The method and device of the invention may also find application in removal of fluid from between edges of the conjunctiva. The fluid may be removed from the conjunctiva in preparation for thermofusion for glaucoma surgery.

The fluid removed may also be, for example, moisture from an intraocular lens (IOL) or other surface. During vitreoretinal (VR) surgery if there has been a YAG capsulotomy and gas introduce, the plastic IOL is at BSS infusion temperature, which is usually room temperature, so the humidity within the eye causes condensation that obscures visibility. The condensate could be effectively removed with the method and device of the invention.

Although not shown, device 200 may comprise a leak free connection 230. In one embodiment, the leak free connection 230 comprises a Luer fitting, also known as a Luer lock or a Luer taper (not shown). As will be readily appreciated by the skilled person, when device 200 comprises a Luer fitting, device 200 may further comprise a male Luer terminal connected via bore 220 to an exit port (not shown) on body 210 or handle 214. The exit port may be disposed in a depression (not shown). The exit port may be closed by a finger or robotic component to prevent flow of fluid or gas from the eye. Removal of the finger or robotic component allows egress of fluid along the needle and through the exit port.

The exit port may be disposed in the single channel bore 220 or may be in a secondary channel.

The method and device of the invention may be referred to as the SLIK WICK method and device.

Device 200 may be manufacturing by disposing the one or more hydrophilic extension or probe 260 on a body 210 comprising a handle 214.

The invention also provides a kit 300 for eye surgery comprising device 200 and optionally further comprising one or more of an aspiration device and instructions for use. In a preferred embodiment, the aspiration device may be or may comprise the Alcon Constellation device, the Synergetics VersaVIT 2.0 Vitrectomy System, any other suitable commercially available device or one or more component thereof.

The following non-limiting examples illustrate the invention. These examples should not be construed as limiting: the examples are included for the purposes of illustration only. The Examples will be understood to represent an exemplification of the invention.

### EXAMPLES

A prototype device 200 was manufactured using microfibers supplied by Dr Shadi Houshyar, Research Fellow, School of Fashion and Textiles, Royal Melbourne Institute of Technology. The prototype device 200 comprising a plurality of microfibres comprising polylactic acid.

The device 200 comprises a body 210 in the form of a Luer lock (25 g needle) tube containing extension or probe 260, in the form of microfibers, that protrude from the tip 212 that is connected to an aspiration line 280 so that it acts like a “flute needle”.

Initial trials showed excellent results with the one or more microfibres readily contacting and capturing subretinal fluid in an eye model with and without aspiration by a hand held syringe for enhanced ease of aspiration.

The initial trials have shown that the use of microfibres provided a device resembling a very fine paintbrush or a woven meshwork of these fibres, where hydrophilic fibres optimise absorption of the sub-retinal fluid because they make contact with the sub-retinal fluid.

In a porcine eye model of a retinal tear, OCT (Optical Coherence Tomography) examination of the retinal tear margin reveals a thick fluid meniscus remains after active aspiration with a standard soft-tip flute needle. The application of a hydrophilic microfibre, as a wick, removed the meniscus. While not wanting to be bound by any one theory, the inventor believes the meniscus was removed due to the fibres conforming to the junction between the retina and the retina pigmented epithelium.

The use of a collection chamber in fluid communication with the extension or probe 260 has the advantage of reducing clogging of the one or more hydrophilic extension or probe and associated adverse effects on the flow of the fluid.

In this specification, the terms “comprises”, “comprising” or similar terms are intended to mean a non-exclusive inclusion, such that an apparatus that comprises a list of elements does not include those elements solely, but may well include other elements not listed.

Throughout the specification the aim has been to describe the invention without limiting the invention to any one embodiment or specific collection of features. Persons skilled in the relevant art may realize variations from the specific embodiments that will nonetheless fall within the scope of the invention.

1. A device for surgery comprising:
a body comprising a handle; and
one or hydrophilic extension or probe,
wherein the device is adapted for removal of hydrophilic fluid.

2. The device of claim 1, wherein the device removes the hydrophilic fluid by one or more of cohesive attraction, adhesion and capillary action; and wherein the removal is at least substantially specific for the hydrophilic fluid.

3. The device of claim 1, wherein the device does not apply any extraneous or additional force or energy.

4. The device of claim 1, wherein the device applies aspiration to assist in removal of the hydrophilic fluid.

5. The device of claim 1, wherein the device comprises a bore to allow passage of air for aspiration of fluid from an eye, optionally the bore comprising an internal bore.

6. The device of claim 5, wherein the bore comprises a diameter of 5; 6; 7; 8; 9; 10; 11; 12; 13; 14; 15; 16; 17; 18; 19; 20; 21; 22; 23; 24; 25; 26; 27; 28; 29; 30; 35; 40 or greater than, gauge.

7. The device of claim 1, for use in eye surgery.

8. A method for removal of hydrophilic fluid, the method comprising:
contacting the hydrophilic fluid with one or more hydrophilic extension or probe to thereby remove the hydrophilic fluid.

9. The method of claim 8, wherein the removal is by one or more of cohesive attraction, adhesion and capillary action, optionally wherein the removal is at least substantially specific for hydrophilic fluid.

10. The method of claim 8, wherein no extraneous or additional force or energy is applied to effect the removal.

11. The method of claim 8, wherein the device applies aspiration to assist in removal of the hydrophilic fluid.

12. The device of claim 1, wherein the fluid is removed by the one or more hydrophilic extension or probe conforming to a junction between the retina and the retinal pigmented epithelium (RPE), optionally the fluid is removed in preparation for thermofusion and/or repair of retinal detachment.

13. The device of claim 1, wherein the fluid removed is from between edges of a conjunctiva, optionally the fluid is removed from the conjunctiva in preparation for thermofusion for glaucoma surgery.

14. The device of claim 1, wherein the fluid removed is moisture from an intraocular lens (IOL) or other surface.

15. The device of claim 14, further comprising a leak free connection, optionally the leak free connection comprising a Luer fitting, a Luer lock or a Luer taper.

16. A kit for surgery comprising the device of claim 1, optionally further comprising an aspiration device and/or instructions for use.

17. The device of claim 1, wherein the one or more of cohesive attraction, adhesion and capillary action is a result of non-covalent bonding, optionally wherein the non-covalent bonding comprises one or more of electrostatic interactions, van der Waals forces, π-effects and hydrophilic effects, optionally the hydrophilic attraction may be charge-polarized and/or capable of hydrogen bonding, and optionally wherein the removal by one or more of cohesive attraction, adhesion and capillary action further comprises one or more of wicking and mopping.

18. The device of claim 1, wherein the one or more hydrophilic extension or probe comprises one or more of polylactic acid (PLA); polyvinyl acetate (PVA) and chitosan, optionally, the chitosan is from a crustacean.

19. The device of claim 18, wherein the one or more hydrophilic extension or probe comprises a large surface area, optionally wherein the one or more hydrophilic extension or probe comprises one or more hydrophilic fibres; mesh; or open weave.

20. The device of claim 1, wherein the one or more hydrophilic extension or probe comprises a diameter from 50 microns to 700 microns; optionally 150 microns to 700 microns.
